(19) United States
(12) Patent Application Publication
(10) Pub. No.: US 2008/0065120 A1
(43) Pub. Date: Mar. 13, 2008

(54) SURGICAL INSTRUMENT, KIT AND METHOD FOR CREATING MATTRESS-TYPE STITCHES

(76) Inventors: Anthony D. Zannis, Fort Wayne, IN (US); Andrew M. Jacobs, Fort Wayne, IN (US); Jerry L. Flax, Lawrenceburg, IN (US); Eric D. Nygaard, West Bend, WI (US); Tyler J. Haskins, Vista, CA (US); Keith M. McGrath, Warsaw, IN (US); Greg M. Penland, Granger, IN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/554,420

(22) Filed: Oct. 30, 2006

Related U.S. Application Data
(60) Provisional application No. 60/732,403, filed on Oct. 31, 2005.

(51) Int. Cl. A61B 17/062; A61B 17/06

(52) U.S. Cl. 606/144; 606/222

(57) ABSTRACT

A surgical kit includes an applicator instrument and a needle assembly. The needle assembly provides two spaced pathways and carries a surgical fastener. One anchor of the surgical fastener is carried by one of the pathways and another anchor of the fastener is carried by the other pathway. A filament connects the two anchors of the fasteners and extends between the pathways. The applicator allows for simultaneous insertion of both anchors of the fastener at spaced locations with the filament extending from both anchors in the tissue and over the surface of the tissue or of a therapeutic implant fixated to the tissue. The needle assemblies may be removed from the applicator. The kit may include several needle assemblies that may be attached to the applicator in series to create a plurality of mattress-type stitches.

### CROSS-REFERENCE TO RELATED APPLICATIONS

Priority is claimed to the following application: U.S. Provisional Patent Application Ser. No. 60/732,403 entitled, “SURGICAL INSTRUMENT, KIT AND METHOD FOR CREATING MATTRESS-TYPE STITCHES,” filed on Oct. 31, 2005 by Anthony D. Zannis, Andrew M. Jacobs, Jerry L. Flax, Eric D. Nygaard, Tyler J. Haskins, Keith M. McGrath and Gregory M. Penland (Docket No. DEP5421USPSP).

### FIELD OF THE INVENTION

The invention relates to surgical devices and procedures in general, and more particularly to surgical devices for delivering fasteners for the repair of tissue defects.

### BACKGROUND OF THE INVENTION

Therapeutic devices have been developed for reinforcement of soft tissue where weakness exists. One example of such a therapeutic device is the RESTORE® orthobiologic implant, available from DePuy Orthopaedics, Inc. of Warsaw, Ind. The DePuy product is indicated for use in reinforcing the rotator cuff. The RESTORE implant is a laminate derived from porcine small intestine submucosa, a naturally occurring extracellular matrix composed primarily of collagenous proteins, that has been cleaned, disinfected, and sterilized. Other biological molecules, such as growth factors, glycosaminoglycans, etc., have also been identified in SIS.

Therapeutic devices such as the RESTORE implant are commonly implanted using a needle, suture and a pair of surgical pliers. The needle is grabbed by surgical pliers and fed through the implant and the tissue. When done arthroscopically, the suturing procedure to fixate the implant may take a substantial amount of time.

Surgical instruments are available for delivering surgical fasteners to a site to hold pieces of soft tissue together. Among the fasteners that have been developed are the “T-type” fastener and the “H-type” fastener. One example of a surgical instrument for deploying T-type and H-type fasteners in tissue is disclosed in U.S. Pat. No. 5,941,439, entitled “Applicator and Method for Deploying a Surgical Fastener in Tissue,” which is incorporated by reference herein in its entirety. The instrument disclosed in that patent has a housing with a trigger for moving a push rod through a needle member to eject a fastener into the tissue. Although such fasteners and such an instrument could be used in fixating a therapeutic device to native soft tissue, they generally are not used to create mattress-type sutures to fixate an implant. Mattress-type suture patterns may be particularly useful in fixating therapeutic devices that comprise a sheet that should be implanted without creases.

Accordingly, a need exists for a surgical instrument and technique for creating mattress- type sutures to fixate a therapeutic implant to native tissue.

### SUMMARY OF THE INVENTION

The present invention provides a surgical instrument and technique for creating mattress- type sutures for fixating a therapeutic implant to native tissue.

In one aspect, the present invention provides a surgical instrument for creating a mattress- type suture stitch comprising a handle assembly, a first pathway, a second pathway spaced from the first pathway and a surgical fastener. The surgical fastener has a first anchor portion carried by the first pathway, a second anchor portion carried by the second pathway, and a filament extending between the first and second anchor portions. The instrument also includes means for simultaneously moving both anchors of the surgical fastener out of the first and second pathways.

In another aspect, the present invention provides a surgical kit for creating mattress-type stitches in a patient. The kit comprises a surgical applicator instrument and a plurality of needle assemblies. Each needle assembly includes a first needle, a second needle and a surgical fastener having a first anchor carried by the first needle, a second anchor carried by the second needle, and a filament extending between the first anchor and the second anchor. Each needle has a pointed distal end. The needle assembly includes a proximal end. Each needle assembly and the surgical applicator instrument include structures for removably mounting the proximal end of the needle assembly to the surgical applicator instrument.

In another aspect, the present invention provides a surgical kit comprising a plurality of pre-packaged needle assemblies. Each needle assembly comprises a first needle, a second needle and a surgical fastener. The first needle includes a pointed distal end portion and a slotted portion. The second needle includes a pointed distal end portion and a slotted portion. The surgical fastener includes a first anchor carried within the slotted portion of the first needle, a second anchor carried within the slotted portion of the second needle, and a filament extending between the first anchor and the second anchor.

In another aspect, the present invention provides a method of creating a mattress-type stitch. A surgical fastener is provided having a first anchor, a second anchor and a filament extending between the first and second anchors. The first and second anchors are simultaneously inserted into tissue at a surgical site at spaced locations. At least a portion of the filament extends over the outer surface of the tissue.

In another aspect, the present invention provides a method of creating a mattress-type stitch. A surgical fastener having a first anchor, a second anchor and a filament extending between the first and second anchors is provided. The first anchor is inserted into tissue at a surgical site. The second anchor is inserted into the tissue at a site spaced from the insertion of the first anchor. At least a portion of the filament extends over the outer surface of the tissue.

In another aspect, the present invention provides a method of fixating a therapeutic soft tissue implant to native tissue with mattress-type stitches. A first surgical fastener having a first anchor, a second anchor and a filament extending between the first and second anchors is provided. The first anchor and the second anchor of the first surgical fastener are simultaneously inserted through the implant and into the native tissue at spaced locations. At least a portion of the filament extends over the outer surface of the implant. A second surgical fastener is also provided. The second surgical fastener has a first anchor, a second anchor and a filament extending between the first and second anchors. The first anchor and the second anchor of the second surgical fastener are simultaneously inserted through the implant and into the native tissue at spaced locations. At least a portion of the filament extends over the outer surface of the implant. The first surgical fastener and second surgical fastener are spaced from each other.

In another aspect, the present invention provides a method of fixating a therapeutic soft tissue implant to native tissue with mattress-type stitches. A first surgical fastener having a first anchor, a second anchor and a filament extending between the first and second anchors is provided. A second surgical fastener having a first anchor, a second anchor and a filament extending between the first and second anchors is also provided. The first anchor of the first surgical fastener is inserted through the therapeutic soft tissue implant and into the native tissue. The second anchor of the first surgical fastener is inserted through the therapeutic soft tissue implant and into the native tissue at a site spaced from the insertion of the first anchor. At least a portion of the filament of the first surgical fastener extends over the outer surface of the therapeutic soft tissue implant. The first anchor of the second surgical fastener is inserted through the therapeutic soft tissue implant and into the native tissue. The second anchor of the second surgical fastener is also inserted through the therapeutic soft tissue implant and into the native tissue at a site spaced from the insertion of the first anchor and spaced from the first surgical fastener. At least a portion of the filament of the second surgical fastener extends over the outer surface of the therapeutic soft tissue implant.

### BRIEF DESCRIPTION OF THE DRAWINGS

FIG. 1 is a perspective view of a first embodiment of a surgical instrument incorporating the principles of the present invention, illustrating two surgical applicators assembled together with two brackets;

FIG. 2 is a longitudinal cross-section of a representative surgical applicator that may be used in the surgical instrument of FIG. 1;

FIG. 2A is a top plan view of a needle assembly that may be attached to the surgical applicator;

FIG. 3 is an enlarged longitudinal cross-section similar to FIG. 2, illustrating the proximal end of the surgical applicator;

FIG. 4 is a perspective view of a portion of the push rod assembly of the surgical applicator of FIGS. 2-3;

FIG. 5 is a perspective view of a portion of the fixed tubular assembly of the surgical applicator of FIGS. 2-3;

FIG. 6 is an enlarged perspective view of the distal end of the elongate tube of the fixed tubular assembly of FIG. 5;

FIG. 7 is a perspective view of a portion of the movable sleeve assembly of the surgical applicator of FIGS. 2-3;

FIG. 8 is an enlarged top plan view of the needle assembly of FIG. 2A;

FIG. 9 is a perspective view of a first embodiment of a surgical fastener that may be used with the present invention;

FIG. 10 is a side elevation of the surgical fastener of FIG. 9;

FIG. 11 is a top plan view of the surgical fastener of FIGS. 9-10;

FIG. 12 is a bottom plan view of the surgical fastener of FIGS. 9-11;

FIG. 13 is a perspective view of one of the surgical anchors of the surgical fastener of FIGS. 9-12.

FIG. 14 is a perspective view of an alternative embodiment of a surgical fastener utilizing the surgical anchor of FIG. 13;

FIG. 15 is a perspective view of another alternative embodiment of a surgical fastener that may be used with the present invention;

FIG. 16 is an elevation of the surgical fastener of FIG. 15;

FIG. 17 is a top plan view of the surgical fastener of FIGS. 15-16;

FIG. 18 is a bottom plan view of the surgical fastener of FIGS. 15-17;

FIG. 19 is a perspective view of one of the surgical anchors of the surgical fastener of FIGS. 15-18;

FIG. 20 is an elevation of another alternative embodiment of a surgical fastener that may be used with the present invention;

FIG. 21 is a top plan view of the needle assembly of FIGS. 2A and 8 packaged for use in a surgical kit;

FIG. 22 is an elevation of an alternative embodiment of a bracket that may be used to hold two surgical applicators together;

FIG. 23 is a top plan view of the bracket of FIG. 22;

FIG. 24 is a longitudinal cross-section of another embodiment of a surgical applicator incorporating the principles of the present invention, illustrating the applicator with its sleeve in a retracted position;

FIG. 24A is a view similar to FIG. 24, illustrating the applicator with its sleeve in an extended position;

FIG. 25 is a top plan view of an alternative embodiment of a needle assembly incorporating the principles of the present invention;

FIG. 26 is a perspective view of a portion of a surgical applicator that may be used with the needle assembly of FIG. 25;

FIG. 27 is a top plan view of another alternative embodiment of a needle assembly incorporating the principles of the present invention;

FIG. 28 is a perspective view of a portion of a surgical applicator that may be used with the needle assembly of FIG. 27;

FIG. 29 is an elevation of a needle assembly connected to an end of a surgical applicator, with the sleeves of the applicator in a retracted position;

FIG. 30 is a view similar to FIG. 29, showing the sleeves in an extended position;

FIG. 31 is a view similar to FIGS. 29 and 30, showing the distal ends of the needle assembly inserted through a therapeutic soft tissue implant, and showing the push rods in an extended position to push the anchors of the surgical fastener into underlying tissue to create a mattress-type stitch;

FIG. 32 is a perspective view of a needle assembly being inserted through a therapeutic soft tissue implant and into an underlying soft surface;

FIG. 33 is a view similar to FIG. 32, illustrating the needle assembly being inserted to a depth sufficient to draw the filament of the surgical fastener taught against the surface of the therapeutic soft tissue implant;

FIG. 34 is a view similar to FIGS. 32 and 33, showing the needle assembly withdrawn from the soft surface, and the remaining mattress-type stitch;

FIG. 35 illustrates the mattress-type stitch created using an instrument incorporating the principles of the present invention;

FIG. 36 is an enlarged diagrammatic cross-section, illustrating anchors of a surgical fastener embedded in soft tissue, and with the filament of the surgical fastener extending up through the soft tissue, through a therapeutic soft tissue implant and across the upper surface of the therapeutic soft tissue implant in a mattress-type stitch;

FIG. 37 is a view similar to FIG. 36, but showing the anchors of the surgical fastener extending past the soft tissue in a mattress-type stitch;

FIG. 38 is a top plan view of another alternative embodiment of a needle assembly incorporating the principles of the present invention; and

FIG. 39 is a top plan view of another alternative embodiment of a needle assembly incorporating the principles of the present invention.

### DETAILED DESCRIPTION

Embodiments of surgical instruments and techniques for fixating therapeutic soft tissue implants to native tissue with mattress-type stitches are described below. The expression “therapeutic soft tissue implants” is intended to mean devices made of both natural and man-made materials that are intended for the repair, support, regeneration or replacement of human muscle, tendons, ligament or cartilage. The expression “native tissue” is intended to mean human tissue, including muscle, tendons, ligaments and cartilage growing, living or occurring naturally in the patient's body. The expression “mattress-type stitch” is intended to mean a length of filament (such as suture, for example) with one portion at one fixed position with respect to native tissue, with a second portion at another fixed position with respect to the native tissue spaced from the fixed position of the first portion, and with a third portion extending between the first and second portions and over the surface of native tissue or an implant.

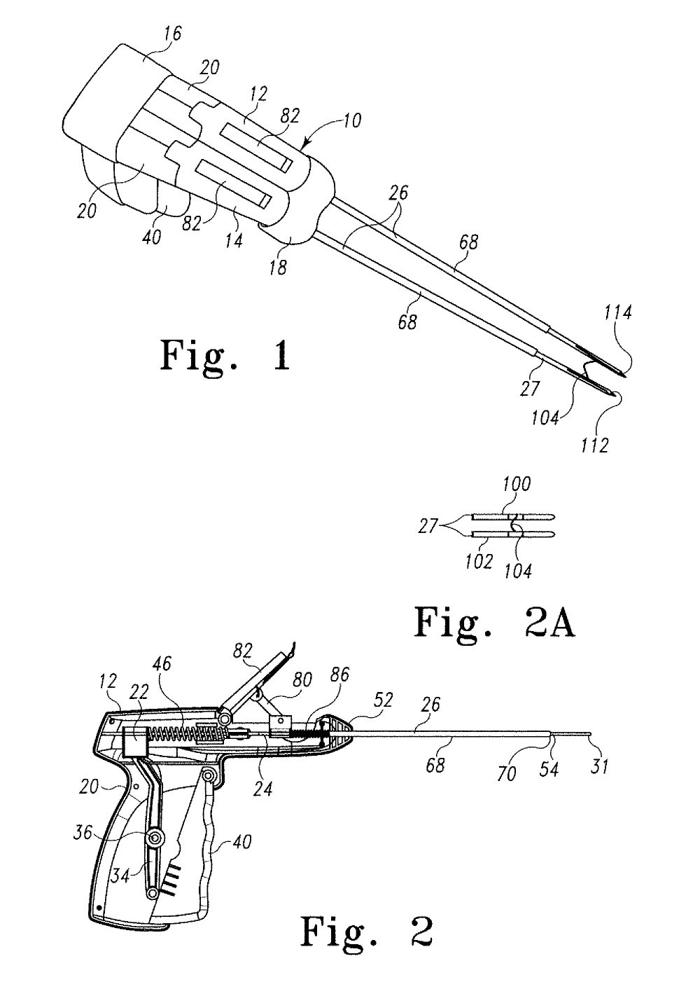

Referring now to FIG. 1, a first embodiment of a surgical instrument incorporating the principles of the present invention is illustrated. The first surgical instrument 10 comprises an assembly of two fastener applicators 12, 14 and brackets 16, 18 for holding the two fastener applicators 12, 14 together in a side-by-side orientation.

The two fastener applicators 12, 14 of the first embodiment are identical. One of these applicators will be described below, and this description should be considered to apply to both applicators. Both applicators may be commercially available applicators. One example of a suitable commercially available surgical applicator is available from the DePuy Mitek, Inc. of Raynham, Mass., part no 228000. A suitable surgical applicator is also disclosed in U.S. Pat. No. 5,941,439, which is incorporated by reference herein in its entirety.

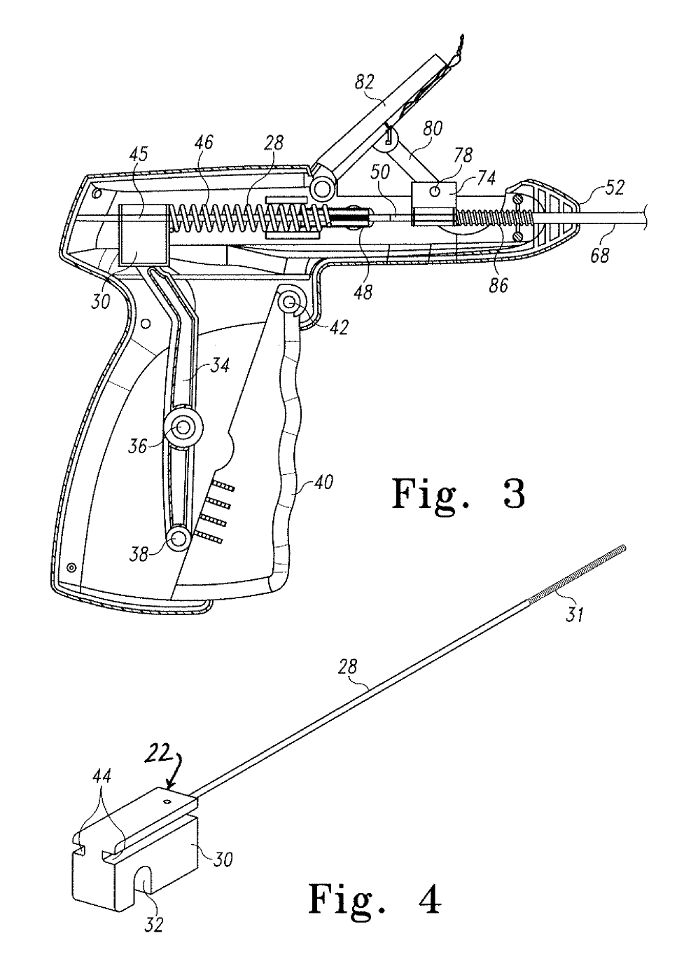

An example of a surgical applicator 12 is illustrated in longitudinal cross-section in FIG. 2. The illustrated surgical applicator includes a handle assembly 20, a push rod assembly 22, a fixed tubular assembly 24 and a movable sleeve assembly 26. A separate needle assembly 27 is also illustrated in FIG. 2A; as described below, the needle assembly 27 may be attached to the fixed tubular assembly 24, with part of the push rod assembly 22 received within the needle assembly 27. As shown in more detail in FIGS. 3-4, the push rod assembly 22 comprises an elongate push rod 28 fixed at or near its proximal end to a slide block 30. The distal end 31 of the push rod 28 is free. The slide block 30 has a slot 32 (see FIG. 4) that receives one end of a pivot member 34. The pivot member 34 is rotatably mounted to the handle assembly 20 at a pivot 36. The opposite end 38 of the pivot member 34 bears against a trigger 40 that is also rotatably mounted to the handle assembly at a pivot 42. The slide block 30 has a pair of longitudinal channels 44 that receive guide rods 45 for mounting the slide block 30 to the handle assembly 20; the slide block 30 may move in a proximal-distal direction on the guide rods. A coil spring 46 biases the slide block 30 in the proximal direction. By pulling the trigger 40, the pivot member 34 is pivoted to push the slide block 30 and the push rod 28 in the distal direction. When the trigger 40 is released, the coil spring 46 pushes the slide block 30 in a proximal direction, thereby retracting the push rod 28.

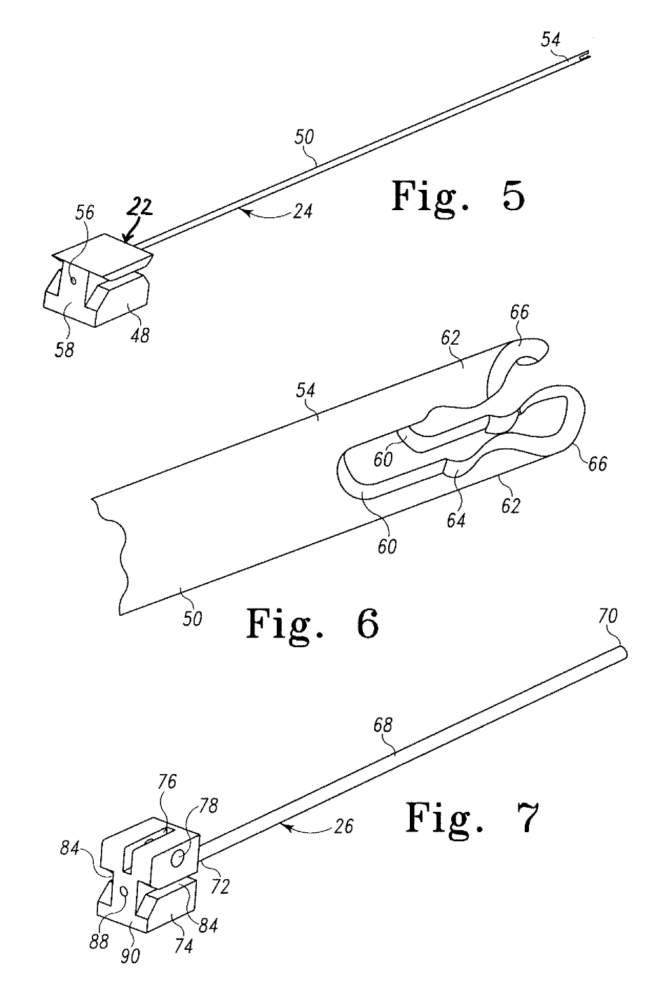

The push rod 28 extends through the coil spring 46 and through a fixed block 48 that is part of the fixed tubular assembly 24. The fixed block 48 is connected to the handle assembly 20 in a fixed position. An elongate hollow tube 50 is attached to and extends out from the fixed block 48, through a hole in the distal end 52 of the handle assembly 20 to a free distal end 54. The fixed tubular assembly 24 is illustrated in FIG. 5. As there shown, the fixed block 48 includes a throughbore 56 extending from its proximal surface 58 through the body of the block 48 to communicate with the elongate hollow tube 50.

The throughbore 56 and the elongate hollow tube 50 are large enough in internal diameter so that the push rod 28 may fit though the bore 56 and tube 50 and slide therein in a proximal- distal direction. The relative lengths of the tube 50 and rod 28 are such that the distal end of the rod 28 extends past the distal end 54 of the tube 50 whether the rod is in a retracted position or an extended position.

The distal end 54 of the tube 50 includes a pair of diametrically-opposed slots 60 which together define a pair of arms 62. Arms 62 are constructed so that they can flex outwardly slightly upon appropriate urging. Each of the slots 60 has a keyway geometry, such that it includes an enlarged opening 64 intermediate to its length. The distal end surfaces 66 of the arms 62 may be beveled.

The tube 50 is received with a hollow elongate sleeve 68 of the movable sleeve assembly 26. As shown in FIG. 7, the sleeve 68 has a free distal end 70 and a proximal end 72 fixed to a movable block 74. The movable block 74 has a slot 76 across its top and a pin 78 that extends transversely through the block 74 and through the slot 76. The pin 78 is pivotally attached to one end of a pivot arm 80 (shown in FIG. 3). The opposite end of the pivot arm 80 is pivotally attached to a lock lever 82. The lock lever 82 is pivotally attached to the handle assembly 20.

The movable block 74 has a pair of longitudinal channels 84 that receive the guide rods 45 for mounting the block 74 to the handle assembly 20; the block 74 may move in a proximal-distal direction on the guide rods. A coil spring 86 (see FIG. 3) biases the block 74 in the proximal direction. By lifting the lock lever 82, the pivot arm 80 is pivoted to pull the block 74 and the sleeve 68 in the proximal direction, thereby retracting the sleeve 68. When the lock lever 82 is pushed downward to the housing, the block 74 and sleeve 68 are pushed in the distal direction relative to the handle assembly 20 to extend the sleeve 68.

The movable block 74 has a throughbore 88 extending from its proximal surface 90 through the body of the block 74 to communicate with the elongate hollow sleeve 68. The throughbore 88 and the elongate hollow sleeve 68 are large enough in internal diameter so that the elongate tube 50 may fit though the bore 88 and sleeve 68 and so that the sleeve 68 and block 74 may slide on the tube 50 in a proximal-distal direction. The relative lengths of the tube 50 and sleeve 68 are such that when the sleeve 68 is retracted, the distal end 54 of the elongate tube 50 is exposed and when the sleeve 68 is extended, the distal end 54 of the elongate tube 50 is received within the sleeve 68. The inner diameter of the sleeve 68 and the outer diameter of the tube 50 are sized so that when the sleeve 68 is extended, its inner walls limit the ability of the arms 62 of the tube 50 to flex.

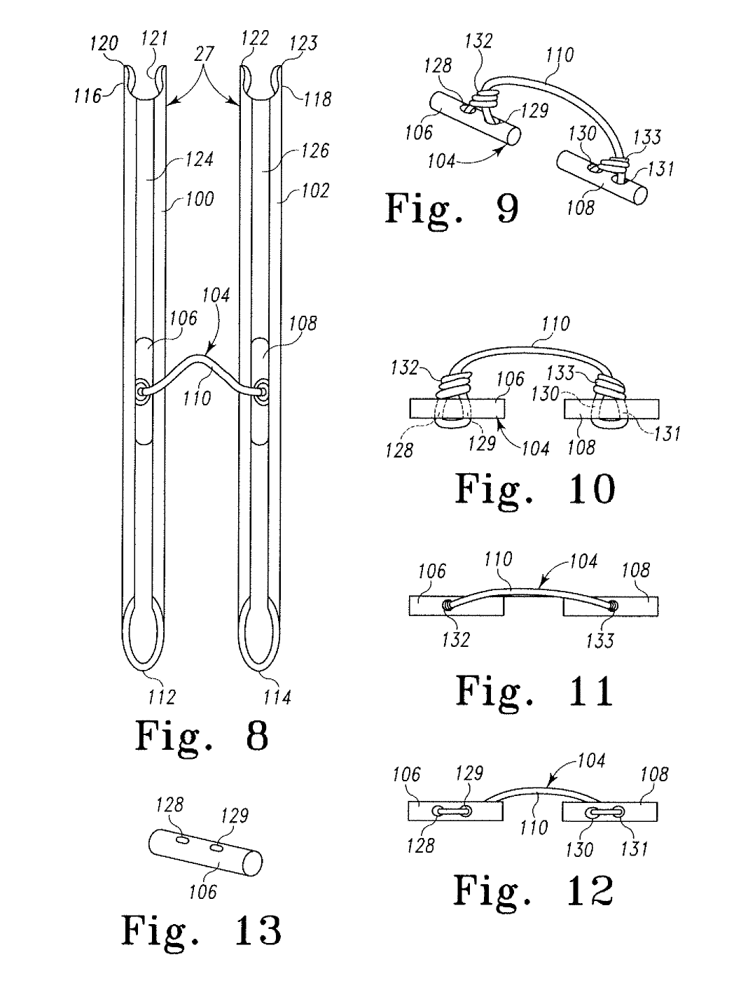

When the sleeve 68 is in the retracted position, the distal end 54 of the elongate tube 50 may be connected to the needle assembly 27. An example of a needle assembly 27 is illustrated in FIGS. 2A and 8. The illustrated needle assembly 27 includes a first hollow elongate needle member 100, a second hollow elongate needle member 102, and a surgical fastener 104. The surgical fastener 104 comprises a first anchor 106, a second anchor 108, and a length of filament 110 connected to each anchor 106, 108 and extending between the two anchors 106, 108.

Each needle member 100, 102 has a distal end 112, 114 and a proximal end 116, 118. The distal end 112, 114 of each needle member 100, 102 terminates in a sharp point. The proximal end 116, 118 of each needle member has a diameter that is substantially the same as the diameter of the distal end 54 of the elongate tube 50. The proximal end 116, 118 of each needle member terminates in a pair of diametrically-opposed tabs 120, 121, 122, 123. The tabs 120, 121, 122, 123 have a configuration that mates with the slots 60 at the distal end 54 of the elongate tube 50. A slot 124, 126 extends along the length of each illustrated needle member 100, 102, although the slots could be formed in the distal portion of each needle member.

The inner diameters of the needle members 100, 102 are sized to receive one of the anchor members 106, 108 of the surgical fastener 104. The connecting length of filament 110 extends out through the slot 124, 126 of each needle member. The anchor members 106, 108 are slidable in the needle members 100, 102. Collar members, as described in U.S. Pat. No. 5,941,439 may be included to releasably hold the fastener 104 in place on the needle assembly 27.

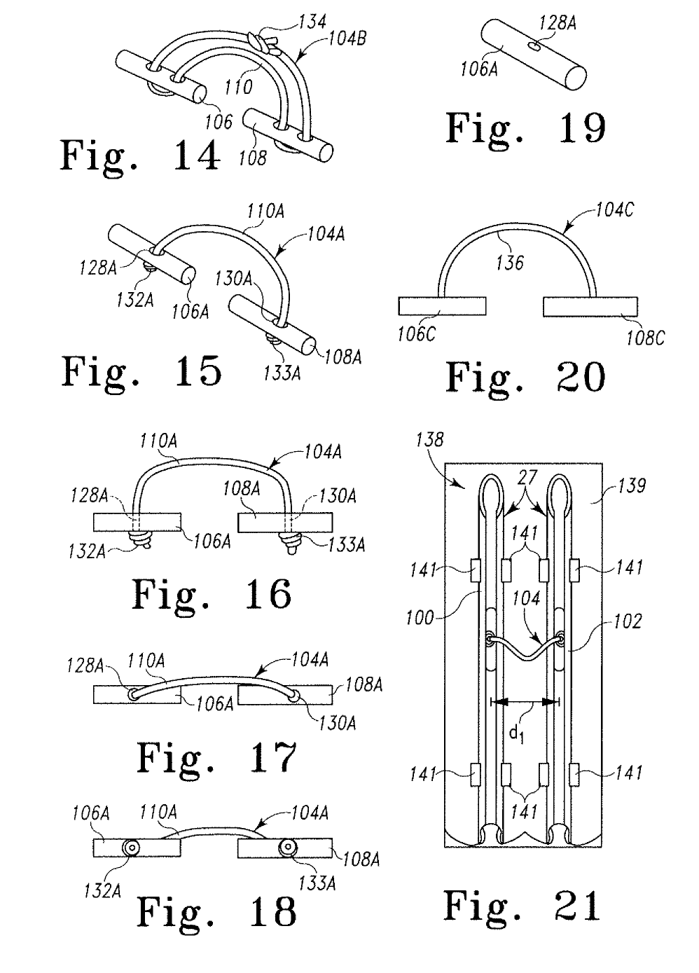

A first embodiment of a surgical fastener that may be used with the present invention is illustrated in FIGS. 9-12. Each of the illustrated surgical anchors 106, 108 has a pair of transverse throughholes 128, 129, 130, 131 (see illustrative anchor in FIG. 13). Part of the filament 110 is threaded through the transverse throughholes 128, 129, 130, 131 and knotted as shown at 132 and 133. FIGS. 15-18 illustrate another embodiment of a surgical fastener 104A, with anchors 106A, 108A having single throughholes 128A, 130A (see illustrative anchor in FIG. 19) through which the filament 11OA passes; in this embodiment, the knots 132A, 133A are on the bottom sides of the anchors 106A, 108A. Another embodiment of a surgical fastener 104B that may be used with the present invention is illustrated in FIGS. 14; this fastener 104B uses the same surgical anchors 106, 108 as the embodiment of FIGS. 9-12, but with the filament 110 doubled and knotted between the anchors 106, 108 as shown at 134. Another embodiment of a surgical fastener 104C that may be used with the present invention is illustrated in FIG. 20. In this surgical fastener 104C, the two anchors 106C and 108C are integral with a single bridge portion 136.

All of the surgical anchors 106, 106A, 108, 108A and the integral fastener 104C may be made of any suitable biocompatible material, and may be made of a bioresorbable material. Examples of materials include polydioxanone and polypropylene. The filament 110 connecting the two anchors 106, 108 may comprise standard suture material. It should be understood that these materials are identified as examples only; the present invention is not limited to any particular material unless expressly called for in the claims.

It should be understood that the above-described surgical fasteners represent examples of surgical fasteners that may be used. U.S. Pat. App. Pub. No. US2005/0187577A1 discloses another anchor system that may be used with the present invention. Accordingly, the present invention is not limited to any particular surgical fastener design unless expressly called for in the claims.

A surgical kit provided for use in the present invention may include needle assemblies 27 with needle members 100, 102 of varying shapes. For example, the needle members 100, 102 of each assembly 27 may both be curved or could be straight. The kit could include needle assemblies 27 of varying lengths. Each needle assembly 27 could be provided in the form of a pre-packaged, sterile assembly, illustrated in FIG. 21 at 138, with the two needle members 100, 102 in a side-by-side relationship at a desired spacing, indicated at d, in FIG. 21. Such a pre-packaged sterile assembly may include a base 139 with appropriate structures such as channels or tabs 141 for holding the two needle members 100, 102 in the desired position. It should be understood that a plurality of such pre-packaged assemblies would typically be provided for use in the implantation of a therapeutic soft tissue implant.

For assistance in determining that the needles 100, 102 have been inserted to the proper depth, each needle may have distance indicia at or near the distal end 112, 114. Examples of such indicia are shown in FIGS. 29-31 at 113 and 115. Alternatively, each needle could be provided with a depth stop; examples of depth stops positioned at the desired maximum depth of for the needles are illustrated at 125 in FIG. 27.

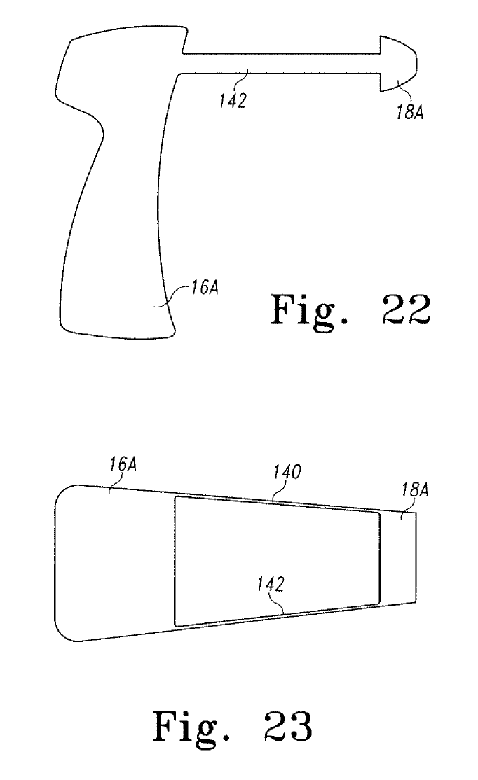

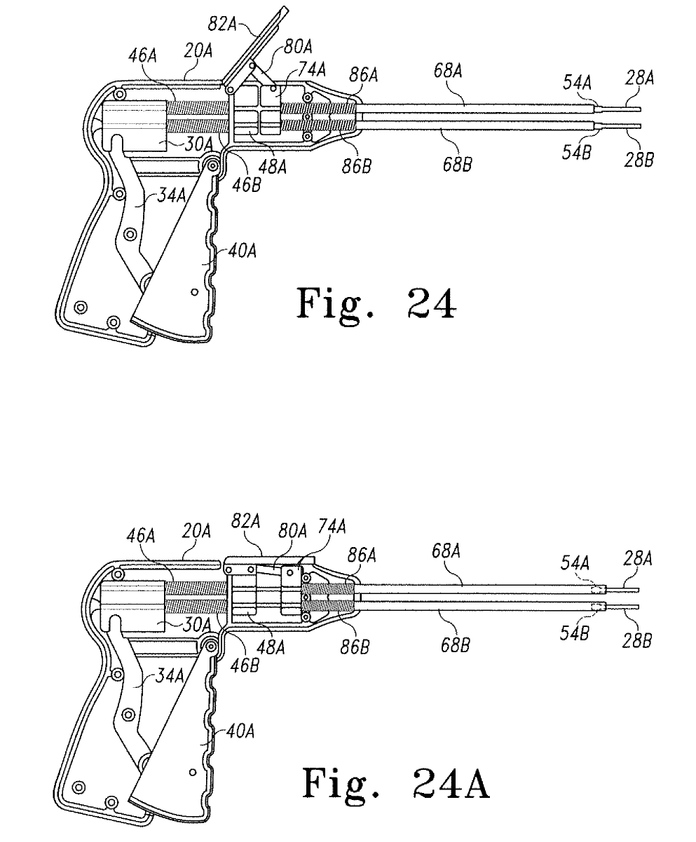

The distal ends 54 of the two tubular members 50 of the two fastener applicators 12, 14 are held together in a side-by-side relationship at a desired spacing by the brackets 16, 18. The first bracket 16 comprises an element formed to closely fit over and hold the proximal ends of two handle assemblies 20 in a side-by-side relationship, as shown in FIG. 1. The second bracket 18 comprises an element formed to closely fit over the distal ends 52 of two handle assemblies. An alternative bracket is illustrated in FIGS. 22-23; in this embodiment, the second or distal bracket portion 18A is connected to the first or proximal bracket portion 16A by side members 140, 142. The brackets may be made out of any suitable surgical grade material such as a surgical grade plastic that has some flexibility for ease of assembly.

The surgical instrument of the present invention could also be constructed as a single instrument with a plurality of push rods, tubes and sleeves. FIG. 24 illustrates such an instrument. The instrument of FIG. 24 has several parts that are similar to those described above for the embodiment of FIGS. 2-3; similar parts are identified with the same reference numeral, followed by letter designations “A” and “B”. The instrument of FIG. 24 has a single handle assembly 20A and no brackets. The push rod assembly in this embodiment comprises a pair of push rods 28A, 28B connected to a single slide block 30A. A single pivot member 34A and a single trigger 40A operate to move the single slide block 30A in a distal direction. A pair of coil springs 46A, 46B bias the slide block 30A in the proximal direction. A single fixed block 48A is connected to two parallel elongate tubes that extend out in a distal direction to distal ends 54A, 54B shaped like the distal end 54 shown in FIG. 6. A pair of parallel movable sleeves 68A, 68B extend over the elongate tubes, and are movable between a retracted position (shown in FIG. 24) where the distal ends 54A, 54b of the tubes are exposed and an extended position (shown in FIG. 24A) where the distal ends 54A, 54B are covered. Both movable sleeves 68A, 68B are connected to a common block 74A that is pivotally connected to a pivot arm 80A. The pivot arm 80A is pivotally connected to a single lock lever 82A that is operable to move the block 74A in a proximal direction. A pair of springs 86A, 86B bias the block 74A in the proximal direction.

In the instrument shown in FIG. 24, the elongate sleeves 68A, 68B are vertically aligned. It should be understood that the instrument could be arranged so that the sleeves are horizontally aligned as well. FIG. 24 is intended to illustrate one way in which multiple anchor delivery mechanisms could be incorporated into a single instrument; it should be appreciated that variations are possible. In addition, it should be understood that additional anchor delivery mechanisms could be included in a single instrument; for example, three delivery mechanisms could be employed. The anchor delivery mechanism could be aligned vertically, horizontally, or in some other pattern.

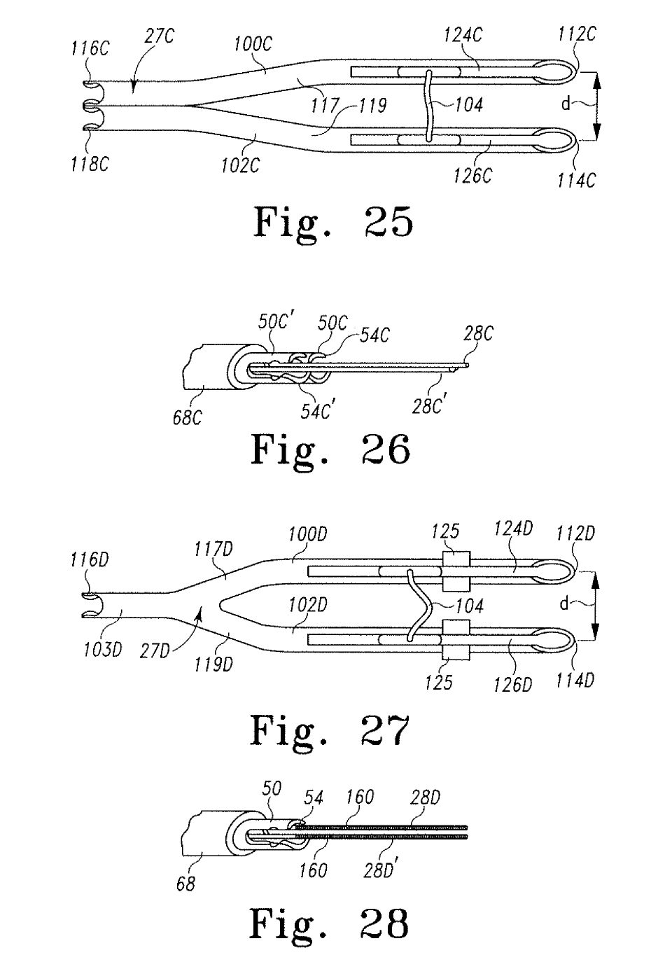

Alternative embodiments of portions of surgical instruments utilizing a single applicator are illustrated in FIGS. 25-28. FIG. 25 illustrates an alternative embodiment of a needle assembly 27C. The needle assembly 27C comprises a first needle member 100C and a second needle member 102C, with a surgical fastener 104 carried by the two needle members 100C, 102C. The distal ends 112C, 114C of the needle members are spaced apart by a distance shown at “d” in FIG. 25, and are sharply pointed. At their proximal ends 116C, 118C, the needle members 100C, 102C are adjacent to each other. Distally diverging portions 117, 119 connect the proximal and distal portions of the needle members 100C, 102C. In this illustrated embodiment, only the distal portions of the needle members 100C, 102C have slots 124C, 126C. FIG. 26 illustrates a portion of a surgical applicator that may be used with the needle assembly 27C of FIG. 25. In the surgical applicator of FIG. 26, two parallel fixed tubes 50C, 50C′ are received within a single movable sleeve 68C. Each fixed tube 50C, 50C′ carries a push rod 28C, 28C′ for receipt in the two needle members 100C, 102C. The proximal ends 116C, 118C of the needle assembly 27C and the distal ends 54C, 54C′ of the tubes 50C, 50C′ have mating tabs and slots as in the first illustrated embodiment. The proximal ends (not shown) of the tubes 50C, 50C′ and sleeve 68C may be connected to blocks as in the first illustrated embodiment, and the proximal ends of the two push rods 28C, 28C′ may both be connected to a single slide block.

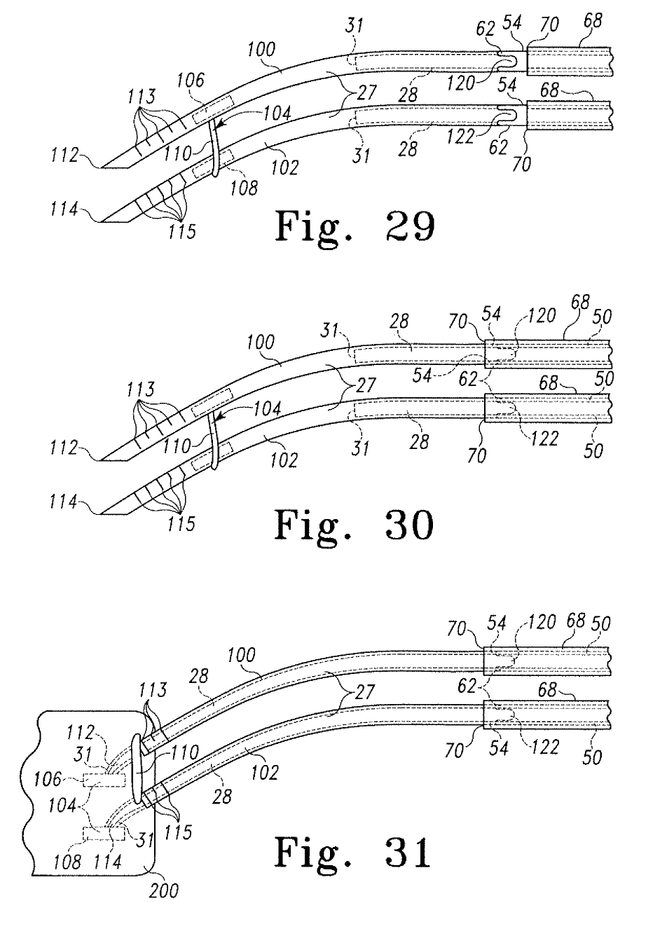

The needle assembly 27D of FIG. 27 comprises a Y-shaped device. Two needle portions 100D, 102D are spaced apart at a distance “d”. These needle portions 100D, 102D include two distally-diverging portions 117D, 119D that meet at their proximal ends at a single, common hollow tubular portion 103D. The proximal end 116D of the hollow tubular portion 103D has tabs to mate with slots at the distal end 54 of the fixed tube 50. FIG. 28 illustrates that the fixed tube 50 may carry two push rods 28D, 28D′ for receipt in the common portion 103D of the needle assembly 27D and that may split off in the needle assembly so that one push rod 28D enters one needle portion 100D and the other push rod 28D′ enters the other needle portion 102D. The two push rods 28D, 28D′ may both be connected to a single slide block. In this embodiment, it may be desirable to provide spacers at the distal end of the tube 50 and proximal end of the hollow tubular portion 103D of the needle assembly 27D to separate and guide the push rods 28D, 28D′ into the appropriate needle portions 100D, 102D.

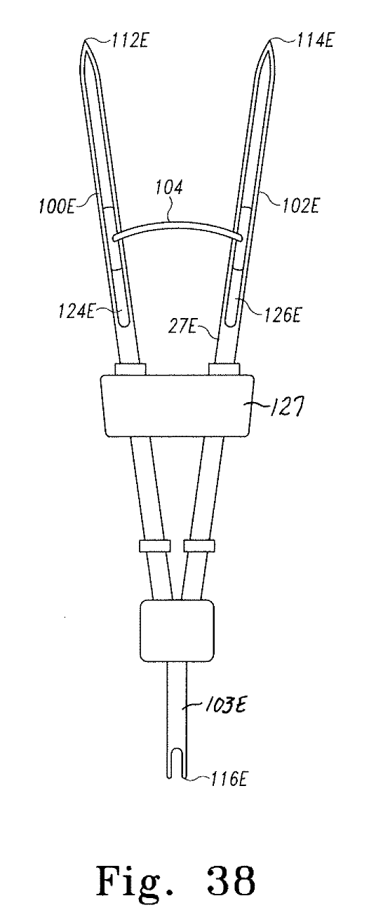

It will also be appreciated that although the instrument of FIG. 24 provides parallel paths for the push rods 28A, 28B, the paths could be convergent, divergent, or the instrument could be modified to allow for adjustment of the angle between the sleeves 68A, 68B. FIG. 38 illustrates a needle assembly 27E that provides distally-divergent paths for the surgical fastener 104. The needle assembly 27E of FIG. 38 also includes a slidable spacer shown at 127. By sliding the spacer 127 in a proximal direction, the distance between the needles 100E, 102E may be increased. By sliding the spacer 127 in a distal direction, the distance between the needles 100E, 102E may be decreased. It will be appreciated that other devices and designs may be used to provide for adjusting the distance between the needles of the needle assemblies.

The illustrated embodiments can be characterized in that a surgical kit embodying these devices would include a plurality of spaced pathways 100, 102, 100C, 102C, 100D, 102D, 100E, 102E for delivering a surgical fastener 104. This plurality of spaced pathways may comprise individual needle members 100, 102, individual needle members 100C, 102C joined at their proximal ends, or individual needle portions 100D, 102D, 100E, 102E joined to a common proximal tubular portion 103D, 103E.

The illustrated embodiments also provide a surgical instrument that provides a means for simultaneously moving both anchors of the surgical fastener out of the pathways. This means for moving the surgical fastener in the illustrated embodiments includes the push rods 28, 28A, 28B, 28C, 28C′, 28D, 28D′ the trigger 40, 40A, the pivot member 34, 34A and a sliding block 30, 30A. The sliding block 30, 30A, pivot member 34, 34A and the trigger 40, 40A may be common elements in a single applicator, as in the embodiment of FIGS. 24 and 24A, or may be a plurality of elements in a plurality of applicators. The push rods 28, 28A, 28B, 28C, 28C′, 28D, 28D′ may be held in a common tube 50 or a plurality of tubes 50A, 50C′ of a single applicator, or may be held in spaced tubes 50 of a plurality of applicators.

For all of the embodiments, the push rods may be made of a flexible material or a shape memory material. For example, a shape memory metal alloy such as nitinol (a nickel-titanium alloy) may be used for the push rods. The push rods could also comprise assemblies, with a distal portion comprising a coil spring for flexibility, as illustrated in FIG. 28 at 160 The components of the surgical applicator and needle assemblies (apart from the surgical fasteners 104) may be made of any standard surgical grade material for surgical instruments, including standard metals and polymers.

To assemble any of the surgical applicators with one of the needle assemblies, the lock lever 82, 82A is lifted to the position shown in FIGS. 2 and 24 to retract the sleeve 68, 68A, 68B. Retracting the sleeve exposes the distal end 54, 54A, 54B, 54C, 54C′ of the fixed tube or tubes 50, 50A, 50C, 50C′. The tabs 120, 121, 122, 123 of the proximal end or ends 116, 118, 116A, 118A, 116B of the needle member or members 100, 102, 100C, 102C, 100D, 102D are inserted into the slots 60 at the distal end 54, 54A, 54B, 54C, 54C′ of the tube or tubes 50, 50A, 50C, 50C′ until the tabs are received in the enlarged openings 64 of the tube or tubes 50, 50A, 50C, 50C′. FIG. 29 illustrates tabs 120, 122 so received between the arms 62 of the tubes 50. To secure the needle assembly to the applicator, the lock lever 82, 82A is then moved downward, to thereby move the block 74, 74A and extend the sleeve 68, 68A in a distal direction to encircle the junctions of the needle assemblies and the fixed tubes. FIGS. 30 and 31 illustrate the sleeves in such an extended position; FIG. 24A illustrates the lock lever 82A and block 74A in the locked positions. With the needle assembly locked to the applicator, the needles may be introduced to the surgical site to puncture a therapeutic soft tissue implant (shown at 200 in FIG. 31) and underlying tissue. The trigger or triggers 40, 40A may then be pulled to push the push rods 28, 28A, 28B, 28C, 28C′, 28D, 28D′ to deliver the anchors 106, 108 of the surgical fastener into the underlying tissue while the length of filament 110 extends over the implant.

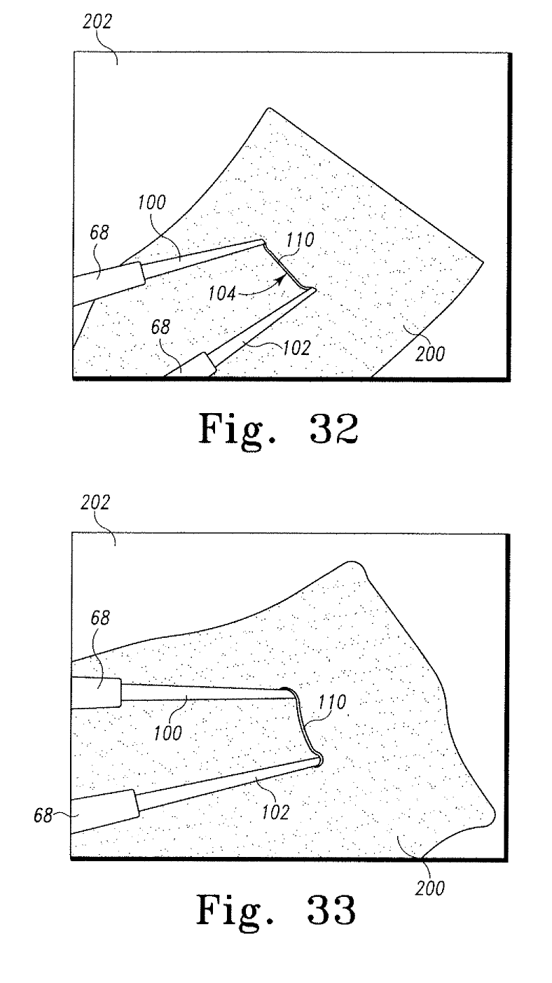

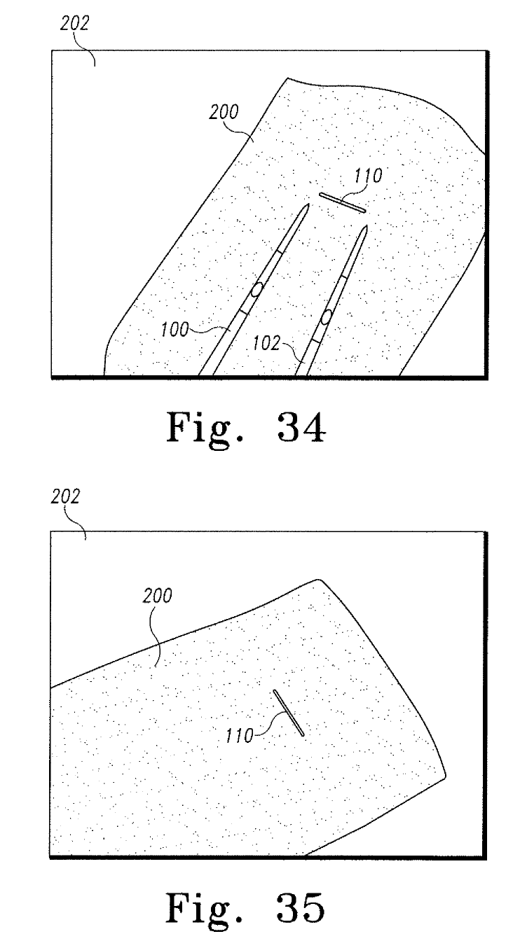

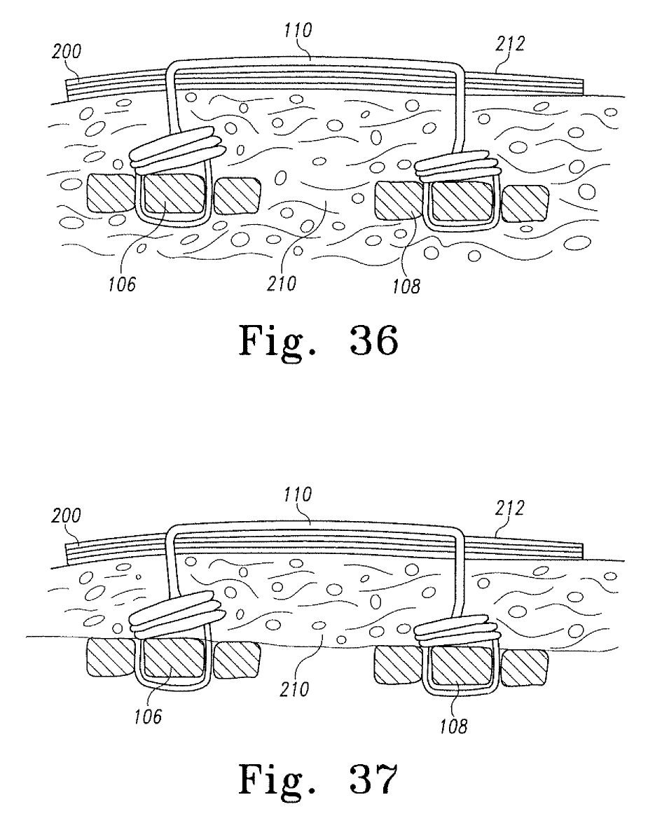

FIGS. 32-35 further illustrate use of the first embodiment to create a mattress-type stitch to hold a therapeutic soft tissue implant 200 to an underlying soft surface 202. As shown in FIG. 32, the sharp distal tips of the needle members 100, 102 are pushed through the therapeutic soft tissue implant 200 and into the underlying soft surface 202. As shown in FIG. 33, the distal tips of the needle members 100, 102 are advanced into the soft surface until the portion of the filament 110 lying on top of the implant 200 is taught. The push rods 28 may then be advanced to push the anchors 106, 108 out of the needle members 100, 102 to be engaged in the underlying soft surface 202. The needle members 100, 102 may then be withdrawn, leaving a taught mattress-type stitch as shown in FIG. 35. It should be understood that FIGS. 32-35 represent an experimental use of the instrument to create a mattress-type stitch in a soft surface; it is anticipated that the same results will be obtained in using the instrument to create such a stitch in vivo. More specifically, it is anticipated that the same results will be obtained in fixating a therapeutic soft tissue implant such as the RESTORE patch to underlying tissue in the area of the rotator cuff of the shoulder. It is also anticipated that the present invention will be useful in open surgical procedures and minimally invasive surgical procedures including arthroscopic procedures. In arthroscopy, it will be appreciated that the needle assembly 27 would be expected to be introduced through a single cannula to the surgical site.

FIG. 36 illustrates a surgical fastener of the type shown in FIGS. 9-12 implanted in a mattress-type stitch configuration. The anchors 106, 108 are embedded within soft tissue 210. The filament 110 extends through the soft tissue 210 and through a laminar therapeutic soft tissue implant 200. The filament 110 extends across the top surface 212 of the therapeutic soft tissue implant 200. The illustrated soft tissue implant 200 is a laminar one. As shown in FIG. 37, the anchors 106, 108 may also extend through the soft tissue 210 if desired.

It is expected that proper fixation of a therapeutic soft tissue implant will require a plurality of appropriately placed mattress-type stitches. Accordingly, a kit embodying the principles of the present invention may include a plurality of needle assemblies. After each mattress-type stitch is created, the surgeon would be expected to withdraw the instrument from the surgical site, remove the used needle assembly, and attach a fresh needle assembly and repeat the process until the therapeutic soft tissue implant is properly fixated.

With surgical fasteners of the type illustrated in FIGS. 9-12 and 14-18 having a fixed length of filament 110, the degree to which the stitch is taught will depend upon the length of the filament, the distance between the needle members or needle portions, and the depth to which the needle members or needle portions may be inserted in the soft tissue. To accommodate different possible depths of insertion, a surgical kit could include fasteners with different lengths of filaments. Alternatively, the instrument may include features that allow the distance between the needle members to be adjusted. A greater distance between the needle members will allow for a taught stitch with a relatively shallow insertion. A smaller distance between the needle members will allow for deeper insertion of the anchors.

It will be appreciated that although the present invention has been described for use in fixation of a therapeutic soft tissue implant to underlying tissue, use is not limited to any particular therapeutic device or implant or to any particular surgical procedure unless expressly called for in the claims. Moreover, it will be appreciated that the principles of the present invention may be applied to other tissue repair procedures that would benefit from the use of a mattress-type stitch, regardless of whether a therapeutic device or implant is used.

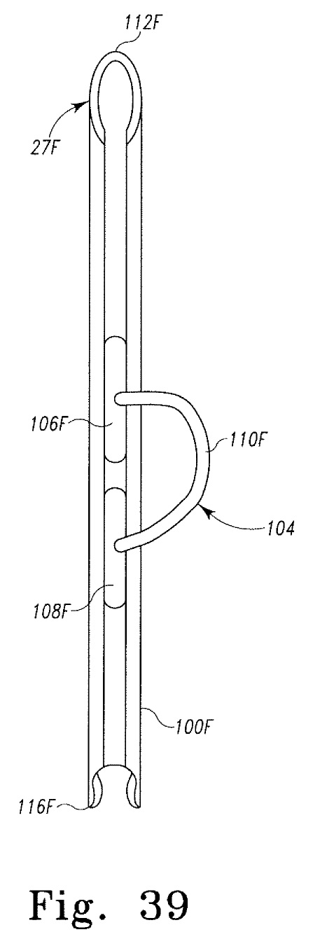

In addition, it will be appreciated that a mattress-type stitch can also be created using a single applicator of the type disclosed in U.S. Pat. No. 5,941,439. As shown in the needle assembly 27F of FIG. 39, both anchor members 106F, 108F could be loaded into a single needle member. The needle member could be inserted to a desired depth in the tissue, and the first anchor member 106F may be pushed into the tissue while the second anchor member 108 is retained on the needle member. The needle member may be withdrawn slightly to the surface of the tissue or implant and reinserted at a small distance from the first insertion point. At the desired depth, the second anchor member 108F may be pushed into the tissue and the needle withdrawn. The filament should rest taught across the surface of the implant or tissue.

Various modifications and additions can be made to the above-described embodiments without departing from spirit of the invention. All such modifications and additions are intended to fall within the scope of the claims unless the claims expressly call for a specific construction. We claim:

1. A surgical instrument for creating a mattress-type suture stitch comprising:
a handle assembly;
a first pathway;
a second pathway spaced from the first pathway;
a surgical fastener having a first anchor portion carried by the first pathway, a second anchor portion carried by the second pathway, and a filament extending between the first and second anchor portions; and
means for simultaneously moving both anchors of the surgical fastener out of the first and second pathways.

2. The surgical instrument of claim 1 wherein:
the first pathway comprises a first elongated hollow needle having a pointed distal end portion, a slotted portion open to the distal end and a proximal portion nearest to the handle assembly; and
the second pathway comprises a second elongated hollow needle having a pointed distal end portion, a slotted portion open to the distal end and a proximal portion nearest to the handle assembly.

3. The surgical instrument of claim 2 wherein the means for simultaneously moving both anchors of the surgical fastener out of the first and second pathways comprises:
a first push rod having a distal end portion adapted for lengthwise movement in the first needle; and
a second push rod having a distal end portion adapted for lengthwise movement in the second needle.

4. The surgical instrument of claim 3 wherein the means for simultaneously moving both anchors of the surgical fastener out of the first and second pathways comprises:
a first slide block mounted in the handle assembly and connected to the first push rod;
a second slide block connected to the second push rod;
a second handle assembly;
wherein the second slide block is mounted in the second handle assembly;
a pivot member mounted in the first handle assembly and having one end in contact with the first slide block and an opposite end;
a trigger mounted on the first handle assembly and in contact with the opposite end of the pivot member;
a second pivot member mounted in the second handle assembly and having one end in contact with the second slide block and an opposite end; and
a second trigger mounted on the second handle assembly and in contact with the opposite end of the second pivot member.

5. The surgical instrument of claim 4 further comprising a bracket holding the first handle assembly and second assembly together in a side-by-side relationship.

6. The surgical instrument of claim 5 further including a first elongate tube and a second elongate tube, the first elongate tube having a first end fixed to the first housing assembly and a second end spaced from the first housing assembly, the second elongate tube having a first end fixed to the second housing assembly and a second end spaced from the second housing assembly, wherein the first push rod extends through the first elongate tube to the first needle and the second push rod extends through the second elongate tube to the second needle member.

7. The surgical instrument of claim 6 wherein the first needle is removably connected to the second end of the first elongate tube and the second needle is removable connected to the second end of the second elongate tube.

8. The surgical instrument of claim 7 further comprising:
a first elongate sleeve extending from the first handle assembly over the first elongate tube and being movable between a retracted position and an extended position, and wherein in the extended position the first elongate sleeve extends over the junction of the first needle and the first elongate tube; and
a second elongate sleeve extending from the second handle assembly over the second elongate tube and being movable between a retracted position and an extended position, and wherein in the extended position the second elongate sleeve extends over the junction of the second needle and the second elongate tube.

9. The surgical instrument of claim 8 wherein the filament of the surgical fastener has a fixed length.

10. The surgical instrument of claim 3 wherein the means for simultaneously moving both anchors of the surgical fastener out of the first and second pathways comprises:
an elongate tube fixed at one end to the handle assembly and having a second end;
a pair of push rods carried within the elongate tube; and
a slide block mounted within the handle assembly and connected to both push rods.

11. The surgical instrument of claim 10 wherein the means for simultaneously moving both anchors of the surgical fastener out of the first and second pathways comprises:
a pivot member mounted in the handle assembly and having one end in contact with the slide block and an opposite end;
a trigger mounted on the handle assembly and in contact with the opposite end of the pivot member;
the trigger being operable to move the pivot member to cause the slide block to move in a distal direction to thereby extend both push rods simultaneously.

12. The surgical instrument of claim 11 wherein the first and second needle are removably connected to the second end of the elongate tube.

13. The surgical instrument of claim 12 further comprising a hollow tube connected to the first and second needle, and wherein the hollow tube is removable connected to the second end of the elongate tube.

14. The surgical instrument of claim 13 wherein the filament of the surgical fastener has a fixed length.

15. The surgical instrument of claim 3 wherein the means for simultaneously moving both anchors of the surgical fastener out of the first and second pathways comprises:
a first elongate tube fixed at one end to the handle assembly and having a second end;
a second elongate tube fixed at one end to the handle assembly and having a second end;
a first push rod carried within the first elongate tube;
a second push rod carried within the second elongate tube; and
a slide block mounted within the handle assembly and connected to both the first and second push rods.

16. The surgical instrument of claim 15 wherein the first needle is removably connected to the second end of the first elongate tube and the second needle is removable connected to the second end of the second elongate tube.

17. The surgical instrument of claim 16 further comprising a sleeve extending over both the first and second elongate tubes, the sleeve being movable between a retracted position and an extended position, and wherein in the extended position the sleeve covers the junction of the first needle and the first elongate tube and the junction of the second needle and the second elongate tube.

18. The surgical instrument of claim 17 wherein the filament of the surgical fastener has a fixed length.

19. A surgical kit for creating mattress-type stitches in a patient, the kit comprising:
a surgical applicator instrument; and
a plurality of needle assemblies, each needle assembly including a first needle, a second needle and a surgical fastener having a first anchor carried by the first needle, a second anchor carried by the second needle, and a filament extending between the first anchor and the second anchor, each needle having a pointed distal end, the needle assembly including a proximal end;
wherein each needle assembly and the surgical applicator instrument include structures for removably mounting the proximal end of the needle assembly to the surgical applicator instrument.

20. The surgical kit of claim 19 wherein the first needle of each needle assembly has a proximal end with structures for removably mounting the first needle to the surgical applicator instrument and wherein the second needle of each needle assembly has a proximal end with structures for removably mounting the second needle to the surgical applicator instrument.

21. The surgical kit of claim 19 wherein the first and second needle share a common proximal end.

22. The surgical kit of claim 19 wherein the filament of each needle assembly has a fixed length.

23. A surgical kit comprising a plurality of pre-packaged needle assemblies, each needle assembly comprising a first needle, a second needle and a surgical fastener, wherein:
the first needle includes a pointed distal end portion and a slotted portion;
the second needle includes a pointed distal end portion and a slotted portion;
the surgical fastener includes a first anchor carried within the slotted portion of the first needle, a second anchor carried within the slotted portion of the second needle, and a filament extending between the first anchor and the second anchor.

24. The surgical kit of claim 23 wherein each needle has a proximal end.

25. The surgical kit of claim 23 wherein the first needle and the second needle are joined at their proximal ends.

26. The surgical kit of claim 23 wherein the filament of each surgical fastener has a fixed length.

27. A method of creating a mattress-type stitch comprising:
providing a surgical fastener having a first anchor, a second anchor and a filament extending between the first and second anchors;
simultaneously inserting the first anchor and the second anchor into tissue at a surgical site, wherein the first anchor and second anchor are simultaneously inserted at spaced locations and at least a portion of the filament extends over the outer surface of the tissue.

28. The method of claim 27 wherein:
the mattress-type stitch is created to fixate a therapeutic soft tissue implant to underlying tissue; and
the step of simultaneously inserting the first anchor and the second anchor into tissue at a surgical site includes inserting the first anchor and second anchor through the therapeutic soft tissue implant at spaced locations, and wherein at least a portion of the filament extends over the outer surface of the therapeutic soft tissue implant.

29. A method of creating a mattress-type stitch comprising:
providing a surgical fastener having a first anchor, a second anchor and a filament extending between the first and second anchors;
inserting the first anchor into tissue at a surgical site;
inserting the second anchor into the tissue at a site spaced from the insertion of the first anchor;
wherein at least a portion of the filament extends over the outer surface of the tissue.

30. The method of claim 29 wherein:
the mattress-type stitch is created to fixate a therapeutic soft tissue implant to underlying tissue;
the step of inserting the first anchor includes inserting the first anchor through the therapeutic soft tissue implant;
the step of inserting the second anchor includes inserting the second anchor through the therapeutic soft tissue implant; and
wherein at least a portion of the filament extends over the outer surface of the therapeutic soft tissue implant.

31. A method of fixating a therapeutic soft tissue implant to native tissue with mattress-type stitches comprising:
providing a first surgical fastener having a first anchor, a second anchor and a filament extending between the first and second anchors;
simultaneously inserting the first anchor and the second anchor of the first surgical fastener through the implant and into the native tissue, wherein the first anchor and second anchors are simultaneously inserted at spaced locations and at least a portion of the filament extends over the outer surface of the implant;
providing a second surgical fastener having a first anchor, a second anchor and a filament extending between the first and second anchors; and
simultaneously inserting the first anchor and the second anchor of the second surgical fastener through the implant and into the native tissue, wherein the first anchor and second anchors are simultaneously inserted at spaced locations and at least a portion of the filament extends over the outer surface of the implant;
wherein the first surgical fastener and second surgical fastener are spaced from each other.

32. A method of fixating a therapeutic soft tissue implant to native tissue with mattress-type stitches comprising:
providing a first surgical fastener having a first anchor, a second anchor and a filament extending between the first and second anchors;
providing a second surgical fastener having a first anchor, a second anchor and a filament extending between the first and second anchors;
inserting the first anchor of the first surgical fastener through the therapeutic soft tissue implant and into the native tissue;
inserting the second anchor of the first surgical fastener through the therapeutic soft tissue implant and into the native tissue at a site spaced from the insertion of the first anchor;
wherein at least a portion of the filament of the first surgical fastener extends over the outer surface of the therapeutic soft tissue implant;
inserting the first anchor of the second surgical fastener through the therapeutic soft tissue implant and into the native tissue; and
inserting the second anchor of the second surgical fastener through the therapeutic soft tissue implant and into the native tissue at a site spaced from the insertion of the first anchor and spaced from the first surgical fastener;
wherein at least a portion of the filament of the second surgical fastener extends over the outer surface of the therapeutic soft tissue implant.
